(19) United States
(12) Patent Application Publication
(10) Pub. No.: US 2025/0375401 A1
(43) Pub. Date: Dec. 11, 2025

(54) PHARMACEUTICAL COMPOSITION CONTAINING DEMETHYLZEYLASTERAL FOR PREVENTION OR TREATMENT OF DISEASES CAUSED BY MUSCLE LOSS

(71) Applicant: SEOUL NATIONAL UNIVERSITY R&DB FOUNDATION, Seoul (KR)

(72) Inventors: HEE YANG, Seoul (KR); SEO HYUN SHIN, Seoul (KR); SEUNG HEE YANG, Seoul (KR); NA RA KIM, Gyeonggi-do (KR); JUNG HAN YOON, Seoul (KR); KI WON LEE, Seoul (KR)

(21) Appl. No.: 18/877,431

(22) PCT Filed: Jun. 19, 2023
(86) PCT No.: PCT/KR2023/008463

§ 371 Date: Dec. 20, 2024

(30) Foreign Application Priority Data
Jun. 21, 2022 (KR) 10-2022-0075558
Jun. 19, 2023 (KR) 10-2023-0078188

(51) Int. Cl. A61K 31/192; A61K 36/37; A61P 21/00

(52) U.S. Cl. CPC A61K 31/192; A61K 36/37; A61P 21/00

(57) ABSTRACT

A method for ameliorating or treating a disease caused by muscle loss includes administering a composition including a compound represented by Formula 1 or a salt thereof to a subject in need thereof. The disease caused by muscle loss may be sarcopenia, muscular dystrophy, myasthenia, myodystrophia, myotonia, muscular hypotonia, muscular weakness, myotonic dystrophy, amyotrophic lateral sclerosis, and/or myasthenia gravis.

### BACKGROUND

### 1. Techinical Field

The present invention relates to a pharmaceutical composition for preventing or treating diseases caused by muscle loss, including demethylzeylasteral.

### 2. Background Art

Skeletal muscle, which makes up 40-50% of the human body, tends to decrease by about 1% after the age of 30 and then declines sharply after the age of 65. Aging causes a decrease in muscle mass and function, leading to a decrease in overall strength and physical performance. ‘Sarcopenia’ refers to the loss of muscle mass and strength caused by a decrease in skeletal muscle, and is closely associated with frailty and osteoporosis.

The number of patients with sarcopenia is gradually increasing: it was 50 million in 2000 and is estimated to exceed 200 million by 2040. Sarcopenia reduces physical performance, increasing the risk of falls, fractures, metabolic disease, and death. In 2000, health-care costs attributable to sarcopenia in the United States were estimated to be approximately $18.5 billion (Janssen et al., 2004). According to the 5th Korean National

Health and Nutrition Survey, the prevalence of sarcopenia in Korea is 14.5% for men and 19.7% for women, and in the age group under 39 years, it is 9.8% for men and 12.5% for women. The Korea National Health and Nutrition Examination Survey also reported that the prevalence of sarcopenia increases with age, yet sarcopenia also occurs in non-elderly populations. This raises the importance of preventing and/or treating sarcopenia.

However, an effective treatment for sarcopenia has not yet been developed. Therefore, there is a need for the development of a pharmaceutical composition that prevents and/or treats sarcopenia. The pathogenesis of sarcopenia is currently understood to include hormonal influences, pro-inflammatory cytokines, decreased myocytes, degradation of muscle proteins, and decreased satellite cell activity. Meanwhile, many pathological conditions characterized by muscle atrophy (sepsis, cachexia, starvation, metabolic acidosis, severe insulinopenia, etc.) have been associated with increased glucocorticoids (Braun et al., 2011).

Previous study has shown that the increased level of cortisol in the blood of the elderly, measured in the afternoon, is associated with decreased resilience of the hypothalamic-pituitary-adrenal axis, and this hypercortisolemia is considered to be one of the factors leading to sarcopenia (Hong et al., 2012). In addition, glucocorticoid, a type of cortisol hormone, whose blood levels increase with aging, adrenal disease, stress, and immunosuppressant medications, are known to cause sarcopenia by activating the expression of genes (atrogin1, MuRF1) associated with muscle atrophy in muscle tissue (Sato et al., 2017).

### SUMMARY

The present invention provides a pharmaceutical composition for preventing or treating a disease caused by muscle loss.

The present invention provides a food composition or feed composition for helping to strengthen or improve muscle strength.

1. A pharmaceutical composition for preventing or treating a disease caused by muscle loss including a compound represented by Formula 1 or a pharmaceutically acceptable salt thereof:

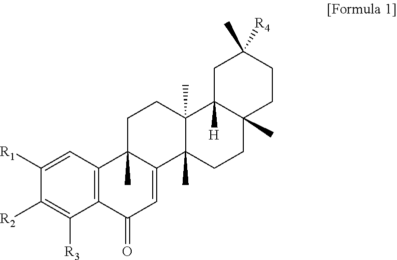

wherein, R₁ is H, OH or C1 to C5 alkyl, R2 is H, OH, OCH₃, OCOCH₃ or C1 to C5 alkyl, R₃ is H, CHO, CH₂OH or C1 to C5 alkyl, and R₄ is CH₂OH, COOH or C1 to C5 alkyl.

2. The pharmaceutical composition of the above 1, wherein the pharmaceutical composition is for administration to a subject selected from the group consisting of a human, a livestock animal, and a pet.

3. The pharmaceutical composition of the above 1, wherein the disease caused by

muscle loss is selected from the group consisting of sarcopenia, muscular dystrophy, myasthenia, myodystrophia, myotonia, muscular hypotonia, muscular weakness, myotonic dystrophy, amyotrophic lateral sclerosis, and myasthenia gravis.

4. The pharmaceutical composition of the above 1, wherein R₁ is H, OH or CH₃, R₂ is H, OH, OCH₃ or OCOCH₃, R₃ is H, CHO, CH₂OH or CH₃, and R₄ is CH₂OH, COOH or CH₃.

5. The pharmaceutical composition of the above 1, wherein the compound represented by Formula 1 is contained in Tripterygium wilfordii, Tripterygium regelii or Tripterygium hypoglaucum.

6. A food composition for helping to prevent muscle loss including a compound represented by Formula 1 or a food-acceptable salt thereof, or an extract of a natural product containing the same.

7. The food composition of the above 6, wherein R₁ is H, OH, or CH₃, R₂ is H, OH, OCH₃, or OCOCH₃, R₃ is H, CHO, CH₂OH, or CH₃, and R₄ is CH₂OH, COOH, or CH₃.

8. The food composition of the above 6, the extract of the natural product is selected from the group consisting of Tripterygium wilfordii extract, Tripterygium regelii extract, and Tripterygium hypoglaucum extract.

9. A feed composition including a compound represented by Formula 1, or a feed-acceptable salt thereof, or an extract of a natural product containing the same.

10. The feed composition of the above 9, wherein R₁ is H, OH or CH₃, R₂ is H, OH, OCH₃ or OCOCH₃, R₃ is H, CHO, CH₂OH or CH₃, and R₄ is CH₂OH, COOH or CH₃.

11. The feed composition of the above 9, wherein the natural product is selected from the group consisting of Tripterygium wilfordii, Tripterygium regelii, and Tripterygium hypoglaucum.

The compound represented by Formula 1 of the present invention is effective in the treatment and prevention of diseases caused by muscle loss, including sarcopenia, muscular dystrophy, myasthenia gravis, and muscle weakness.

The compound represented by Formula 1 of the present invention could act as an antagonist of the glucocorticoid receptor to regulate the transcriptional mechanism of genes involved in muscle differentiation or muscle degradation.

The compound represented by Formula 1 of the present invention could transform myoblast into myotubes, promote muscle differentiation, and inhibit muscle degradation.

### BRIEF DESCRIPTION OF THE DRAWINGS

FIG. 1 shows the morphology of myotubes from a mouse muscle cell line treated with demethylzeylasteral at concentrations of 0.5, 1 or 2 μM and dexamethasone at a concentration of 100 μM as observed by microscopy.

FIG. 2 shows myotube formation and myocyte differentiation in myoblast cell line treated with demethylzeylasteral at a concentration of 0.5 or 1 μM and dexamethasone at a concentration of 100 μM, observed using immunofluorescence staining and DAPI staining.

FIG. 3 is a graph showing myocyte differentiation index quantified by dividing the total number of MHC+nuclei by the total number of nuclei in myoblast cell line treated with 0.5 or 1 μM demethylzeylasteral and 100 μM dexamethasone.

FIG. 4 is a graph quantifying the number of nuclei in myoblast cell line treated with 0.5 or 1 μM demethylzeylasteral and 100 μM dexamethasone using DAPI staining.

FIG. 5 shows bands representing the level of expression of total MHC, a biomarker for myocyte differentiation in late stage: MuRF-1, a biomarker for muscle degradation; and b-actin, a loading control.

FIG. 6 shows two bands representing the level of expression of p-AKT/AKT, a biomarker for the muscle protein synthesis, in a mouse muscle cell line treated with 1 μM concentration of demethylzeylasteral and 100 μM concentration of dexamethasone, in duplicate.

FIG. 7 is a graph quantifying the level of expression of p-AKT/AKT, the biomarker for the muscle protein synthesis, in a mouse muscle cell line treated with 1 μM demethylzeylasteral and 100 μM dexamethasone, in two replicates.

### DETAILED DESCRIPTION OF THE EMBODIMENTS

Hereinafter, the present invention will be described in detail.

The present invention relates to a pharmaceutical composition for preventing or treating a disease caused by muscle loss, including a compound represented by Formula 1 or a pharmaceutically acceptable salt thereof (hereinafter, collectively referred to as ‘the compound represented by Formula 1’).

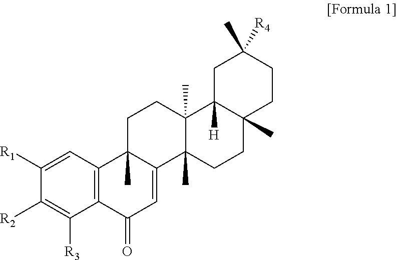

In Formula 1, R₁ is H, OH, or C1 to C5 alkyl, R₂ is H, OH, OCH₃, OCOCH₃, or C1 to C5 alkyl, R₃ is H, CHO, CH₂OH, or C1 to C5 alkyl, and R₄ is CH₂OH, COOH, or C1to C5 alkyl.

In Formula 1, it is preferable that R₁ is H, OH, or CH₃, R₂ is H, OH, OCH₃, or OCOCH₃, R₃ is H, CHO, CH₂OH, or CH₃, and R₄ is CH₂OH, COOH, or CH₃.

More preferably, the compound represented by Formula 1 has the structure of Formula 2 below.

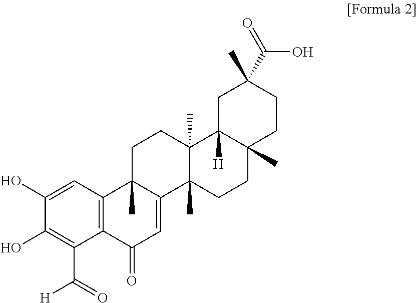

The compound represented by Formula 2 is demethylzeylasteral. It is a type of triterpenoid compound with a molecular formula of C₂₉H₃₆O₆ and a molecular weight of 480.60, and is also named 12-oxodendrobane or (2R,4aS,6aR,6aS,14aS,14bR)-9-formyl-10,11-dihydroxy-2,4a,6a,6a,14a-pentamethyl-8-oxo-1,3,4,5,6,13,14,14b-octahydropicene-2-carboxylic acid.

Demethylzeylasteral could be synthesized chemically or isolated from natural products such as Tripterygium wilfordii Hook. f., Tripterygium regelii, and Tripterygium hypoglaucum. When isolating from natural products, solvents such as chloroform, dichloromethane, ethyl acetate, DMSO, or acetone can be used.

The pharmaceutical composition of the present invention has a preventive and/or therapeutic effect on a disease caused by muscle loss. The disease caused by muscle loss include diseases caused by poor protein intake, lack of exercise, or poor exercise habits; diseases caused by hormonal deficiencies associated with aging; acute or chronic diseases such as diabetes, infectious diseases, and cancer; and secondary conditions to degenerative diseases such as spinal stenosis. The disease caused by muscle loss include, for example, sarcopenia, muscular dystrophy, myasthenia, myodystrophia, myotonia, muscular hypotonia, muscular weakness, myotonic dystrophy, amyotrophic lateral sclerosis (ALS), myasthenia gravis and the like.

The pharmaceutically acceptable salt of a compound of the present invention refers to a salt that is pharmaceutically acceptable as defined in the present invention and has the desired pharmacological activity of the parent compound.

The pharmaceutically acceptable salt may be, for example, acid addition salts or metal salts.

Acid addition salts can be formed from inorganic acids such as hydrochloric, nitric, phosphoric, sulfuric, hydrobromic, hydroiodic, nitrous or phosphorous acid, and non-toxic organic acids such as aliphatic mono-and di-carboxylates, phenyl-substituted alkanoates, hydroxy alkanoates and alkanedioates, aromatic acids, aliphatic and aromatic sulfonic acids. These pharmaceutically non-toxic salts include sulfates, pyrosulfates, bisulfates, sulfites, bisulfites, nitrates, phosphates, mono hydrogen phosphates, dihydrogen phosphates, metaphosphates, pyrophosphates, chlorides, bromides, iodides, fluorides, acetates, propionates, decanoates, caprylates, acrylates, formates, isobutyrates, caprates, heptanoates, propylates, oxalates, malonates, succinates, suberates, sebacates, fumarates, maleates, butyne-1,4-dioate, hexane-1,6-dioate, benzoates, chlorobenzoates, methylbenzoate, dinitrobenzoate, hydroxybenzoates, methoxybenzoates, phthalates, terephthalate, benzenesulfonate, toluenesulfonate, chlorobenzenesulfonate, xylenesulfonate, phenylacetate, phenylpropionate, phenylbutyrate, citrate, lactate, β-hydroxybutyrate, glycolate, malate, tartrate, methanesulfonate, propanesulfonate, naphthalene-1-sulfonate, naphthalene-2-sulfonate or mandelate. For example, acid addition salts can be obtained by dissolving a compound in an excess of aqueous acid solution and precipitating the salt using a hydrated organic solvent, such as methanol, ethanol, acetone or acetonitrile.

The metal salts may be sodium, potassium or calcium salts. Metal salts can be prepared using a base, for example, alkali metal or alkaline earth metal salts can be obtained by dissolving the compound in an excess of alkali metal hydroxide or alkaline earth metal hydroxide solution, filtering the non-dissolved compound salt, and evaporating and/or drying the filtrate.

These salts could be prepared by conventional chemical methods from compounds having a basic or acidic moiety, and a corresponding acid or base.

The pharmaceutical composition of the present invention may be formulated according to conventional methods in oral formulations such as pills, granules, tablets, capsules, suspensions, emulsions, syrups, aerosols: topicals: suppositories; and sterile injectable solutions.

Examples of carriers, excipients, and diluents that may be included in the composition include, but are not limited to, lactose, dextrose, sucrose, dextrin, maltodextrin, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, and mineral oils. When formulated, it may be prepared using commonly used, but not limited to, excipients or diluents such as fillers, extenders, binders, wetting agents, disintegrants, and surfactants.

Examples of solid formulations for oral administration include, but are not limited to, tablets, pills, powders, granules, capsules and the like, and these solid formulations are prepared by mixing with at least one excipient selected from starch, calcium carbonate, sucrose or lactose, gelatin, and the like. Further, in addition to simple excipients, lubricants such as magnesium stearate and talc may also be used.

Examples of liquid formulations for oral administration include, but are not limited to, suspensions, emulsions, syrups, and the like, and may include a variety of excipients, such as wetting agents, sweeteners, flavors, and preservatives, in addition to the commonly used simple diluents of water or liquid paraffin. Examples of formulations for parenteral administration include sterile aqueous solutions, non-aqueous solutions, suspensions, emulsions, lyophilized preparations, and suppositories. Examples of non-aqueous solvents and suspensions include propylene glycol, polyethylene glycol, vegetable oils including olive oil, and injectable esters such as ethyl oleate. Bases for the suppositories may be witepsol, macrogol, tween 61, cacao butter, Laurin, or glycerogelatin.

The pharmaceutical composition of the present invention is administered in a pharmaceutically effective amount. As used herein, “pharmaceutically effective amount” refers to an amount sufficient to treat a disease at a reasonable benefit/risk ratio applicable to medical treatment, and the effective dose level may be determined based on factors including the type and severity of the patient's condition, the activity of the drug, sensitivity to the drug, time of administration, route of administration and excretion rate, duration of treatment, concomitant medications, and other factors well known in the medical field. The pharmaceutical composition of the present invention may be administered individually or in combination with other conventional therapeutic agents. When administered in combination, the pharmaceutical compositions may be administered sequentially or simultaneously with other conventional therapeutic agents. The pharmaceutical composition may be administered in a single dose or multiple doses. Taking all of the above factors into account, it is important to administer an amount that can achieve the maximum effect with the minimum amount without any adverse side effects, and the amount may be readily determined by a person of ordinary skill in the art.

In the present invention, the effective amount of the pharmaceutical composition may vary depending on the age, gender, and weight of the patient. However, the above amount is not intended to limit the scope of the present invention in any way, as it may be increased or decreased depending on the route of administration, severity of the disease, gender, weight, age and the like.

A subject to which the pharmaceutical composition is administered is not particularly limited, but may preferably be mammals including domestic animal, human, and the like. In the present invention, “domestic animal” refers to any animal useful in human life that has been domesticated and improved by mankind from wildlife.

Examples of domestic animal include, but are not limited to, cattle, horses, mules, donkeys, goats, mountain goats, cotton sheep, deer, pigs, rabbits, and poultry, and examples poultry include, but are not limited to, chickens, turkeys, ducks, ostriches, geese, and quails, as long as they are suitable for raising to obtain livestock products. “Livestock products” refer to meat, milk, eggs, honey and their processed products, raw skin (including raw fur), raw wool, and other livestock products as defined in Article 2 (3) of the Livestock Act, as prescribed by the Ministry of Agriculture and Forestry.

The subject to which the pharmaceutical composition is administered may be pets, including livestock, poultry, or fish.

Further, the present invention provides a food composition including a compound represented by the Formula 1 or a food-acceptable salt thereof.

The food composition of the present invention includes all forms such as functional food, nutritional supplement, health food, and food additives.

The food composition may contain conventional food additives, and their suitability as food additives is determined by the standards and criteria for such items in accordance with the general rules and general test methods of Food Additives Codex approved by Ministry of Food and Drug Safety in Korea, unless otherwise specified.

Items listed in the “Food Additives Codex” include, for example, chemical synthetics such as ketones, glycine, potassium citrate, nicotinic acid and cinnamic acid: natural additives such as persimmon color, licorice extract, crystalline cellulose, kaoliang color and guar gum: mixed formulations such as L-sodium glutamate formulations, alkali agents for noodles, preservative formulations and tar color formulations.

The food composition of the present invention may be provided in the form of a tablet, and the tablet may be formed by mixing the composition with excipients, binders, disintegrants, and other additives, granulating in a conventional manner, and then compressing with a lubricant or the like, or compressing the mixture directly. Furthermore, the food composition in tablet form may contain flavorings or the like as desired.

The food composition of the present invention may be provided in capsule form, for example, such as a hard capsule prepared by mixing the composition with additives such as excipients and filling it into a conventional hard capsule, or a soft capsule prepared by mixing the composition with additives such as excipients and filling it into a capsule base such as gelatin. The soft capsule may contain colorants, preservatives, plasticizers such as glycerin or sorbitol, as desired.

The food composition of the present invention may be provided in pellet form, and may be prepared by mixing the composition with an excipient, a binder, a disintegrant, and/or the like, and molding the mixture using a conventionally known method, and, if necessary, may be coated with white sugar or other coating agents, or the surface may be coated with a material such as starch or talc.

The food composition of the present invention may be provided in granular form, which may be prepared by conventional methods known in the art by mixing the composition with excipients, binders, disintegrating agents, and the like, and flavorings, if desired.

The food composition may be, but is not limited to, a beverage, meat, chocolate, food product, confectionery, pizza, ramen, other noodles, chewing gum, candy, ice cream, alcoholic beverage, vitamin complexes, and dietary supplements.

The present invention may provide a food composition for reducing body fat, controlling blood sugar, controlling blood pressure, and anti-obesity including a compound represented by Formula 1 or a food-acceptable salt thereof.

The food composition may further include a food-acceptable dietary supplement in addition to the active ingredient.

The present invention also provides a feed composition including a compound represented by Formula 1, or a feed-acceptable salt thereof, or an extract of a natural product including the same.

As used herein, the term “feed” refers to any natural or artificial diet, meal, or the like, or any ingredient of said meal, which is intended for or adapted to be eaten, consumed, and digested by an animal.

More specifically, the present invention may provide a feed composition for preventing muscle loss and/or inhibiting muscle protein degradation, including a compound represented by Formula 1 or a feed-acceptable salt thereof.

In the present invention, the feed may be a feed for reptiles, fish, birds, or mammals. Preferably, it may be a feed for livestock or aquatic organisms that are suitable for breeding due to their wild nature being acclimated, and can contribute to increasing the income of farmers, as defined in Article 2, Paragraph 1 of the Livestock Industry Act and Article 2, Subparagraphs of the Enforcement Decree of the same Act.

Formula 1 of the present invention may have substituents as shown in Table 1 below:

TABLE 1

| No. | R₁ | R₂ | R₃ | R₄ |
|---|---|---|---|---|
| 1 | H | H | H | CH₃ |
| 2 | H | H | H | CH₂OH |
| 3 | H | H | H | COOH |
| 4 | H | H | CH₃ | CH₃ |
| 5 | H | H | CH₃ | CH₂OH |
| 6 | H | H | CH₃ | COOH |
| 7 | H | H | CHO | CH₃ |
| 8 | H | H | CHO | CH₂OH |
| 9 | H | H | CHO | COOH |
| 10 | H | H | CH₂OH | CH₃ |
| 11 | H | H | CH₂OH | CH₂OH |
| 12 | H | H | CH₂OH | COOH |
| 13 | H | OH | H | CH₃ |
| 14 | H | OH | H | CH₂OH |
| 15 | H | OH | H | COOH |
| 16 | H | OH | CH₃ | CH₃ |
| 17 | H | OH | CH₃ | CH₂OH |
| 18 | H | OH | CH₃ | COOH |
| 19 | H | OH | CHO | CH₃ |
| 20 | H | OH | CHO | CH₂OH |
| 21 | H | OH | CHO | COOH |
| 22 | H | OH | CH₂OH | CH₃ |
| 23 | H | OH | CH₂OH | CH₂OH |
| 24 | H | OH | CH₂OH | COOH |
| 25 | H | OCH₃ | H | CH₃ |
| 26 | H | OCH₃ | H | CH₂OH |
| 27 | H | OCH₃ | H | COOH |
| 28 | H | OCH₃ | CH₃ | CH₃ |
| 29 | H | OCH₃ | CH₃ | CH₂OH |
| 30 | H | OCH₃ | CH₃ | COOH |
| 31 | H | OCH₃ | CHO | CH₃ |
| 32 | H | OCH₃ | CHO | CH₂OH |
| 33 | H | OCH₃ | CHO | COOH |
| 34 | H | OCH₃ | CH₂OH | CH₃ |
| 35 | H | OCH₃ | CH₂OH | CH₂OH |
| 36 | H | OCH₃ | CH₂OH | COOH |
| 37 | H | OCOCH₃ | H | CH₃ |
| 38 | H | OCOCH₃ | H | CH₂OH |
| 39 | H | OCOCH₃ | H | COOH |
| 40 | H | OCOCH₃ | CH₃ | CH₃ |
| 41 | H | OCOCH₃ | CH₃ | CH₂OH |
| 42 | H | OCOCH₃ | CH₃ | COOH |
| 43 | H | OCOCH₃ | CHO | CH₃ |
| 44 | H | OCOCH₃ | CHO | CH₂OH |
| 45 | H | OCOCH₃ | CHO | COOH |
| 46 | H | OCOCH₃ | CH₂OH | CH₃ |
| 47 | H | OCOCH₃ | CH₂OH | CH₂OH |
| 48 | H | OCOCH₃ | CH₂OH | COOH |
| 49 | CH₃ | H | H | CH₃ |
| 50 | CH₃ | H | H | CH₂OH |
| 51 | CH₃ | H | H | COOH |
| 52 | CH₃ | H | CH₃ | CH₃ |
| 53 | CH₃ | H | CH₃ | CH₂OH |
| 54 | CH₃ | H | CH₃ | COOH |
| 55 | CH₃ | H | CHO | CH₃ |
| 56 | CH₃ | H | CHO | CH₂OH |
| 57 | CH₃ | H | CHO | COOH |
| 58 | CH₃ | H | CH₂OH | CH₃ |
| 59 | CH₃ | H | CH₂OH | CH₂OH |
| 60 | CH₃ | H | CH₂OH | COOH |
| 61 | CH₃ | OH | H | CH₃ |
| 62 | CH₃ | OH | H | CH₂OH |
| 63 | CH₃ | OH | H | COOH |
| 64 | CH₃ | OH | CH₃ | CH₃ |
| 65 | CH₃ | OH | CH₃ | CH₂OH |
| 66 | CH₃ | OH | CH₃ | COOH |
| 67 | CH₃ | OH | CHO | CH₃ |
| 68 | CH₃ | OH | CHO | CH₂OH |
| 69 | CH₃ | OH | CHO | COOH |
| 70 | CH₃ | OH | CH₂OH | CH₃ |
| 71 | CH₃ | OH | CH₂OH | CH₂OH |
| 72 | CH₃ | OH | CH₂OH | COOH |
| 73 | CH₃ | OCH₃ | H | CH₃ |
| 74 | CH₃ | OCH₃ | H | CH₂OH |
| 75 | CH₃ | OCH₃ | H | COOH |
| 76 | CH₃ | OCH₃ | CH₃ | CH₃ |
| 77 | CH₃ | OCH₃ | CH₃ | CH₂OH |
| 78 | CH₃ | OCH₃ | CH₃ | COOH |
| 79 | CH₃ | OCH₃ | CHO | CH₃ |
| 80 | CH₃ | OCH₃ | CHO | CH₂OH |
| 81 | CH₃ | OCH₃ | CHO | COOH |
| 82 | CH₃ | OCH₃ | CH₂OH | CH₃ |
| 83 | CH₃ | OCH₃ | CH₂OH | CH₂OH |
| 84 | CH₃ | OCH₃ | CH₂OH | COOH |
| 85 | CH₃ | OCOCH₃ | H | CH₃ |
| 86 | CH₃ | OCOCH₃ | H | CH₂OH |
| 87 | CH₃ | OCOCH₃ | H | COOH |
| 88 | CH₃ | OCOCH₃ | CH₃ | CH₃ |
| 89 | CH₃ | OCOCH₃ | CH₃ | CH₂OH |
| 90 | CH₃ | OCOCH₃ | CH₃ | COOH |
| 91 | CH₃ | OCOCH₃ | CHO | CH₃ |
| 92 | CH₃ | OCOCH₃ | CHO | CH₂OH |
| 93 | CH₃ | OCOCH₃ | CHO | COOH |
| 94 | CH₃ | OCOCH₃ | CH₂OH | CH₃ |
| 95 | CH₃ | OCOCH₃ | CH₂OH | CH₂OH |
| 96 | CH₃ | OCOCH₃ | CH₂OH | COOH |
| 97 | OH | H | H | CH₃ |
| 98 | OH | H | H | CH₂OH |
| 99 | OH | H | H | COOH |
| 100 | OH | H | CH₃ | CH₃ |
| 101 | OH | H | CH₃ | CH₂OH |
| 102 | OH | H | CH₃ | COOH |
| 103 | OH | H | CHO | CH₃ |
| 104 | OH | H | CHO | CH₂OH |
| 105 | OH | H | CHO | COOH |
| 106 | OH | H | CH₂OH | CH₃ |
| 107 | OH | H | CH₂OH | CH₂OH |
| 108 | OH | H | CH₂OH | COOH |
| 109 | OH | OH | H | CH₃ |
| 110 | OH | OH | H | CH₂OH |
| 111 | OH | OH | H | COOH |
| 112 | OH | OH | CH₃ | CH₃ |
| 113 | OH | OH | CH₃ | CH₂OH |
| 114 | OH | OH | CH₃ | COOH |
| 115 | OH | OH | CHO | CH₃ |
| 116 | OH | OH | CHO | CH₂OH |
| 117 | OH | OH | CHO | COOH |
| 118 | OH | OH | CH₂OH | CH₃ |
| 119 | OH | OH | CH₂OH | CH₂OH |
| 120 | OH | OH | CH₂OH | COOH |
| 121 | OH | OCH₃ | H | CH₃ |
| 122 | OH | OCH₃ | H | CH₂OH |
| 123 | OH | OCH₃ | H | COOH |
| 124 | OH | OCH₃ | CH₃ | CH₃ |
| 125 | OH | OCH₃ | CH₃ | CH₂OH |
| 126 | OH | OCH₃ | CH₃ | COOH |
| 127 | OH | OCH₃ | CHO | CH₃ |
| 128 | OH | OCH₃ | CHO | CH₂OH |
| 129 | OH | OCH₃ | CHO | COOH |
| 130 | OH | OCH₃ | CH₂OH | CH₃ |
| 131 | OH | OCH₃ | CH₂OH | CH₂OH |
| 132 | OH | OCH₃ | CH₂OH | COOH |
| 133 | OH | OCOCH₃ | H | CH₃ |
| 134 | OH | OCOCH₃ | H | CH₂OH |
| 135 | OH | OCOCH₃ | H | COOH |
| 136 | OH | OCOCH₃ | CH₃ | CH₃ |
| 137 | OH | OCOCH₃ | CH₃ | CH₂OH |
| 138 | OH | OCOCH₃ | CH₃ | COOH |
| 139 | OH | OCOCH₃ | CHO | CH₃ |
| 140 | OH | OCOCH₃ | CHO | CH₂OH |
| 141 | OH | OCOCH₃ | CHO | COOH |
| 142 | OH | OCOCH₃ | CH₂OH | CH₃ |
| 143 | OH | OCOCH₃ | CH₂OH | CH₂OH |
| 144 | OH | OCOCH₃ | CH₂OH | COOH |

Hereinafter, the present invention will be described in detail with reference to Examples.

### EXAMPLE

Dimethylzeylasteral used in the Examples was 10 mg of Demethylzeylasteral (CAS No. 107316-88-1, product number 28595, Cayman Chemical, USA) dissolved in DMSO.

### Example 1

### Effect of Demethylzeylasteral on Myotube Formation

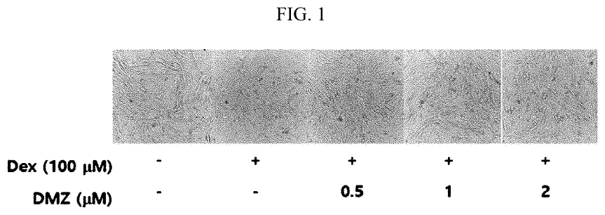

To confirm that demethylzeylasteral (DMZ) has an effect on myotube formation in a dexamethasone (Dex)-induced cell model, the following experiments were performed. Specifically, myoblast cell line C2C12 from mouse were seeded in 6 cm dishes and incubated for 72 hours. Then, the media were replaced to differentiation media to differentiate myoblasts into myotubes for 48 hours. After myotubes were formed, they were teated with 100 μM dexamethasone and 0.5, 1, or 2 μM demethylzeylasteral for 48 hours while replacing the differentiation media every 24 hours. The differentiation media were suctioned off, and the dishes in which the cells were incubated were washed three times with PBS. Then, the cells, i.e., myotubes were fixed with 4% formaldehyde, and the morphology of the myotubes was observed using a microscope (FIG. 1).

### Example 2

Effect of Demethylzeylasteral on Myocyte Differentiation To confirm that demethylzeylasteral has an effect on myocyte differentiation in a

dexamethasone-induced cell model, the following experiments were performed. Specifically, myoblast cell line C2C12 from mouse were seeded in 6 cm dishes and incubated for 72 hours. Then, the media were replaced to differentiation media to differentiate myoblasts into myotubes for 48 hours. After myotubes were formed, they were teated with 100 μM dexamethasone and 0.5, or 1 μM demethylzeylasteral for 72 hours while replacing the differentiation media every 24 hours. The differentiation media were suctioned off, and the dishes in which the cells were incubated were washed three times with PBS. Then, the cells were fixed with 4% formaldehyde, permeabilized with PBS containing 0.1% Triton-X 100 for 10 minutes, and blocked with PBS containing 3% bovine serum albumin for 1 hour. The cells were treated with total MHC antibodies, a biomarker for myocyte differentiation, followed by Alexa 488-conjugated secondary antibodies, and teated with DAPI for staining nuclei. The ratio of MHC+ nuclei to the total number of nuclei was calculated and plotted as a differentiation index, and the total number of nuclei stained with DAPI was also counted and plotted.

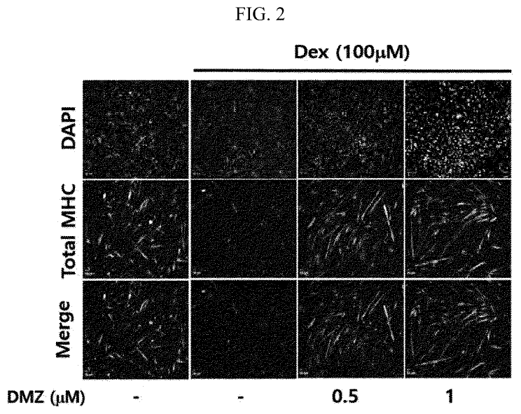

FIG. 2 shows myotube formation and myocyte differentiation in myoblast cell line treated with demethylzeylasteral at a concentration of 0.5 or 1 μM and dexamethasone at a concentration of 100 μM, observed using immunofluorescence staining and DAPI staining.

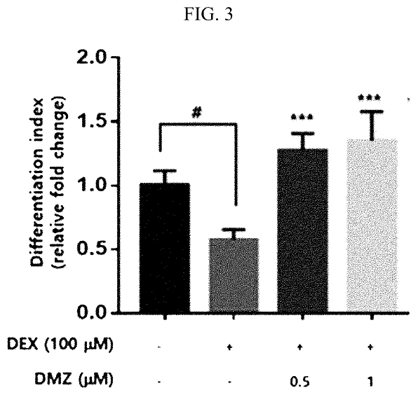

FIG. 3 is a graph showing myocyte differentiation index quantified by dividing the total number of MHC+ nuclei by the total number of nuclei in myoblast cell line treated with 0.5 or 1 μM demethylzeylasteral and 100 μM dexamethasone. Myocyte differentiation, which was significantly reduced by dexamethasone treatment, was significantly increased and restored by demethylzeylasteral treatment.

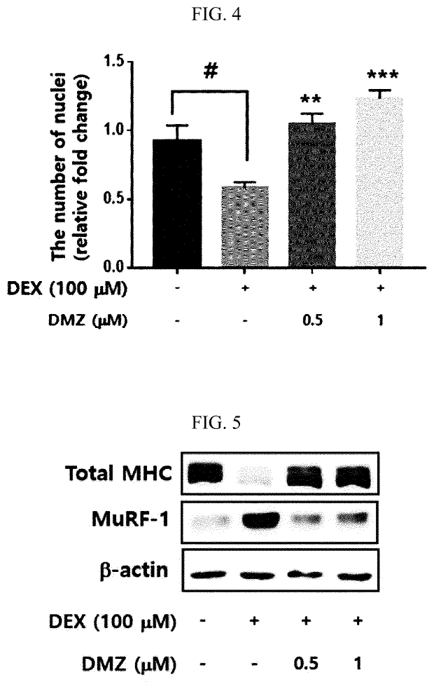

FIG. 4 is a graph quantifying the number of nuclei in myoblast cell line treated with 0.5 or 1 μM demethylzeylasteral and 100 μM dexamethasone using DAPI staining. The number of nuclei reduced by dexamethasone treatment was significantly increased by demethylzeylasteral treatment, confirming the myocyte-protective effect of demethylzey lasteral.

### Example 3

### Effect of Demethylzeylasteral on Promoting Myocyte Differentiation and Inhibiting Muscle Degradation

To confirm that demethylzeylasteral has an effect on promoting myocyte differentiation and inhibiting muscle degradation in a dexamethasone-induced cell model, the following experiments were performed. Specifically, myoblast cell line C2C12 from mouse were seeded in 6 cm dishes and incubated for 72 hours. Then, the media were replaced to differentiation media to differentiate myoblasts into myotubes for 72 hours. After myotubes were formed, they were teated with 100 μM dexamethasone and 0.5 or 1 μM demethylzeylasteral for 48 hours while replacing the differentiation media every 24 hours. Then, the cells were scraped using RIPA buffer. Proteins were then extracted by centrifugation at 13000 g for 10 min, and quantified using protein assay reagent kits (Bio-Rad Laboratories, Hercules, CA, USA). The extracted proteins were electrophoresed using SDS polyacrylamide gels and then transferred to nitrocellulose blotting membranes (GE Healthcare Life Science, Amersham, UK). The membranes were then blocked with 5% skim milk for 1 hour and treated with primary antibodies for total MHC, MuRF-1 and b-actin, diluted 1:1000, for 16 hours in a 4° C. refrigerator. The primary antibodies were washed with TBS-T buffer for 3 times, 10 minutes each time, and then treated with secondary antibodies diluted 1:5000 in 5% skim milk for 1 hour. The secondary antibodies were then washed with TBS-T buffer for 5 times, 8 minutes each time. The luminescence of each biomarker was then detected using ECL detection kit and imagined in Chemidoc.

FIG. 5 shows bands representing the level of expression of total MHC, a biomarker for myocyte differentiation in late stage: MuRF-1, a biomarker for muscle degradation; and b-actin, a loading control. The expression of total MHC was significantly decreased by dexamethasone treatment, whereas it was not decreased by dexamethasone and demethylzeylasteral treatment, indicating significantly increased expression. The expression of MuRF-1 was increased by dexamethasone treatment, whereas it was not increased by dexamethasone and demethylzeylasteral treatment, indicating significantly reduced expression.

### Example 4

### Effect of Demethylzeylasteral on Muscle Protein Synthesis

To confirm that demethylzeylasteral has an effect on muscle protein synthesis in a dexamethasone-induced cell model, the following experiments were performed. Specifically, myoblast cell line C2C12 from mouse were seeded in 6 cm dishes and incubated for 72 hours. Then, the media were replaced to differentiation media to differentiate myoblasts into myotubes for 48 hours. After myotubes were formed, they were teated with 100 μM dexamethasone and 1 μM demethylzeylasteral for 48 hours while replacing the differentiation media every 24 hours. Then, the cells were scraped using RIPA buffer. Proteins were then extracted by centrifugation at 13000 g for 10 min, and quantified using protein assay reagent kits (Bio-Rad Laboratories, Hercules, CA, USA). The extracted proteins were electrophoresed using SDS polyacrylamide gels and then transferred to nitrocellulose blotting membranes (GE Healthcare Life Science, Amersham, UK). The membranes were then blocked with 5% skim milk for 1 hour and treated with primary antibodies forp-AKT, AKT, b-actin diluted 1:1000, for 16 hours in a 4° C. refrigerator. The primary antibodies were washed with TBS-T buffer for 3 times, 10 minutes each time, and then treated with secondary antibodies diluted 1:5000 in 5% skim milk for 1 hour. The secondary antibodies were then washed with TBS-T buffer for 5 times, 8 minutes each time. The luminescence of each biomarker was then detected using ECL detection kit, imagined in Chemidoc, and the levels of protein expression were quantified using Image J software (National Institutes of Health, Bethesda, MD, USA).

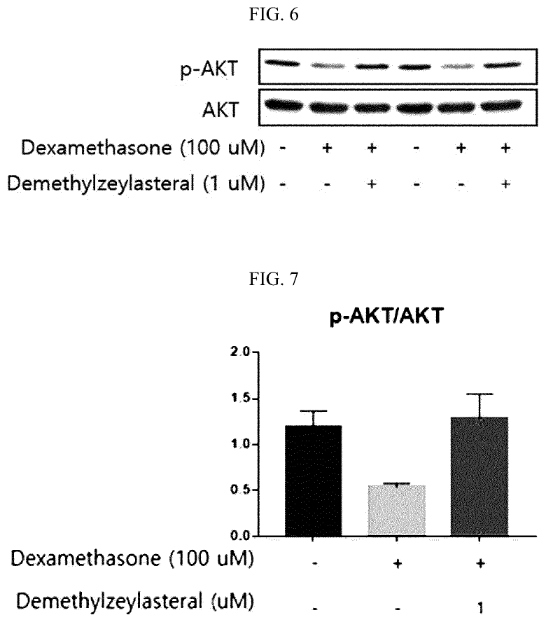

FIG. 6 shows two bands representing the level of expression of p-AKT/AKT, a biomarker for the muscle protein synthesis, in a mouse muscle cell line treated with 1 μM concentration of demethylzeylasteral and 100 μM concentration of dexamethasone, in duplicate.

FIG. 7 is a graph quantifying the level of expression of p-AKT/AKT, the biomarker for the muscle protein synthesis, in a mouse muscle cell line treated with 1 μM demethylzeylasteral and 100 μM dexamethasone, in two replicates.

1. A method for ameliorating or treating a disease caused by muscle loss, the method comprising:
administering a composition comprising a compound represented by Formula 1 or a salt thereof to a subject in need thereof: 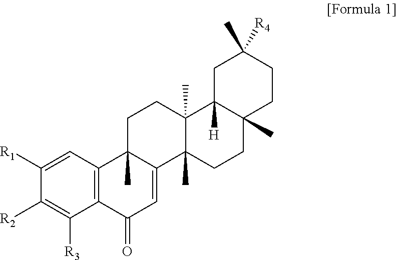
( wherein R₁ is H, OH or C1 to C5 alkyl, R₂ is H, OH, OCH₃, OCOCH₃ or C1 to C5 alkyl, R₃ is H, CHO, CH₂OH or C1 to C5 alkyl, and R₄ is CH₂OH, COOH or C1 to C5 alkyl.

2. The method of claim 1, wherein the subject is selected from the group consisting of a human, a livestock animal, and a pet.

3. The method of claim 1, wherein the disease caused by muscle loss is selected from the group consisting of sarcopenia, muscular dystrophy, myasthenia, myodystrophia, myotonia, muscular hypotonia, muscular weakness, myotonic dystrophy, amyotrophic lateral sclerosis, and myasthenia gravis.

4. The method of claim 1, wherein R₁ is H, OH or CH₃, R₂ is H, OH, OCH₃ or OCOCH₃, R3 is H, CHO, CH₂OH or CH₃, and R₄ is CH₂OH, COOH or CH₃.

5. The method of claim 1, wherein the compound represented by Formula 1 is contained in an extract of Tripterygium an extract of wilfordii, an extract of Tripterygium regelii and/or an extract of Tripterygium hypoglaucum.

6-11 (canceled)

12. The method of claim 1, wherein the composition is a pharmaceutical composition, and the salt is a pharmaceutically acceptable salt thereof.

13. The method of claim 1, wherein the composition is a food composition, and the salt is a food-acceptable salt thereof.

14. The method of claim 1, wherein the subject is a human.

15. The method of claim 1, wherein the subject is a livestock animal or a pet.
